(19) United States
(12) Patent Application Publication
(10) Pub. No.: US 2015/0197433 A1
(43) Pub. Date: Jul. 16, 2015

(54) Compositions and methods for treating toilets

(71) Applicant: Roy Martin, Downers Grove, IL (US)

(72) Inventor: Roy Martin, Downers Grove, IL (US)

(73) Assignee: Integrated Chemical Technologies, Inc., Downers Grove, IL (US)

(21) Appl. No.: 14/544,447

(22) Filed: Jan. 7, 2015

Related U.S. Application Data
(60) Provisional application No. 61/964,768, filed on Jan. 14, 2014.

(51) Int. Cl. C02F 1/50; A61L 2/23; C02F 1/66; A61L 2/16

(52) U.S. Cl. CPC C02F 1/50; A61L 2/16; A61L 2/23; C02F 1/66; C02F 2101/16

(57) ABSTRACT

The invention relates to compositions and methods for treating a toilet to reduce or eliminate offensive odors such as ammonia resulting from the enzymatic decomposition of urea. Use of the compositions and methods substantial reduce or eliminate offensive odors for an extended period of time even when temperatures of the treated water is greater than or equal to 80° F.

### BACKGROUND

There is a need for reducing or all together eliminating the strong ammonia odor resulting from of urine accumulation in toilet holding tank and urinals. More specifically the decomposition of urea and corresponding increase in pH results in the release of offensive concentrations of ammonia released from urinals and portable toilets.

Surprisingly, this object is achieved through the use of the compositions and methods disclosed, even under conditions in which the urine and fecal contaminated water is heated to greater than or equal to 80° F., more preferred 90° F., and most preferred 100° F. for a period of at least 5 days, more preferably 6 days and most preferred 7 days.

The use of hydroxamic acid derivatives and pH buffers achieves the object of the invention. The hydroxamic acid derivatives and pH buffers can be used alone or combined with other substances.

### BRIEF DESCRIPTION OF THE INVENTION

In the first aspect of the invention, the invention is a composition for treating a toilet comprising at least one hydroxamic acid derivative and a pH buffer. The toilet treatment may further comprise other ingredients exemplified by a colorant, fragrance, sequestants, chelants, dispersants, surfactants and the like.

Hydroxamic acid derivatives have the general formula:

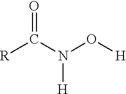

wherein R is selected from at least one: 1 to 6 carbon substituted or un-substituted alkyl group, 6 carbon substituted or un-substituted aromatic group, and 4 to 5 carbon substituted or un-substituted heterocyclic group.

Non-limiting examples of substitution groups include: amine, hydroxyl, chlorine, thiol, carboxyl, methyl, ethyl, propyl, butyl, benzyl and the like.

Hydroxamic acid derivatives can be in the form a liquid, gel, or solid. The typical concentration of hydroxamic acid derivatives in the aqueous solution in the toilet holding tank is from 20 ppm to 2000 ppm. The hydroxamic acid derivative can be applied as a single dose treatment and/or in periodic doses. A single dose may be applied as a toilet treatment comprising at least one hydroxamic acid derivative, a biocide, and pH buffer. The toilet treatment may further comprise fragrance, colorant, chelant, sequestrant and surfactants. A single dose solid composition may further comprise effervescing additives to enhance dispersion of the ingredients when added to the portable toilets holding tank.

Periodic dosing may comprise a dispenser that periodically applies the composition comprising hydroxamic acid derivative, or a solid tablet that slowly dissolves when contacted with urine or water in a urinal. The dosing of hydroxamic acid derivatives will typically range between about 0.002 to 20 ppm based on the amount of water and/or urine applied to the urinal.

The hydroxamic acid derivatives are produced by reaction between an organic acyl donor with a source of hydroxamic acid in an aqueous solution. Non-limiting examples of organic acyl donors include: amino acids exemplified by phenylalanine, serine, alanine, glycine, histidine, threonine, leucine, arginine, aspartic acid, glutamic acid, cysteine, proline and the like. Other non-limiting examples of acyl donors include: acetic acid, acetyl chloride, acetic anhydride, sodium acetate, nicotinic acid, salicylic acid, acetylsalicylic acid and the like.

The hydroxamic acid derivatives are preferably produced by forming an aqueous solution comprising a source of hydroxamic acid and combining with an organic acyl donor by adding the organic acyl donor to the aqueous solution comprising a source of hydroxamic acid. By adding the organic acyl donor to the aqueous solution comprising a source of hydroxamic acid, a stoichiometric excess of hydroxamic acid is sustained, thereby favoring the formation of the N-acyl derivatives in favor of O-acyl derivatives.

The pH buffer is used to buffer the pH of the aqueous solution in the toilet holding tank and/or the urine or water applied to the urinal from between 2.0 to 7.2, more preferably 4.0 to 7.0, and most preferred 5.0 to 6.8. Non-limiting examples of pH buffers include: monosodium phosphate, disodium phosphate, citric acid, citrate, succinic acid, fumaric acid, sodium bisulfate, sulfamic acid, boric acid, borates, sodium bicarbonate and the like. The pH buffering program may combine two or more pH buffers to achieve the desired control of pH in the aqueous solution in the toilet holding tank. The buffering of the pH reduces or prevents the conversion of ammonium into the volatile ammonia.

Solid toilet treatments can be produced by drying the composition comprising an aqueous solution of hydroxamic acid derivatives. The aqueous solution of hydroxamic acid derivative can be dried using any convenient means known to those skilled in the art. Non-limiting examples include spray drying, film drying, wiped filmed drying and the like. One preferred method is to combine the aqueous solution of hydroxamic acid derivative with amorphous silicon dioxide exemplified by Hi-Sil™ 200 series (available by PPG Industries), allowing the amorphous silicon dioxide to absorb the said solution, and drying. Non-limiting examples of suitable drying equipment may include vacuum rotary drum dryers, vacuum tray dryers, rotary dryers and the like.

The aqueous composition can alternatively be treated with an alkali to adjust the pH if needed to produce a salt form of the composition. The addition of alkali neutralizes excess acidity released during formation of some hydroxamic acid derivative converting the acids to salts suitable for drying. However, in the case of some composition exemplified by glycine hydroxamate, and other amino acid based hydroxamates, no neutralization is required to produce a dry salt form.

The solid toilet treatments can be in the form of a tablet, or can be in the form of a powder or granules and packaged in a single use disposable water soluble package, illustrated by the non-limiting examples: a water soluble polyvinyl alcohol, or water soluble paper. The package may be in the form of a pouch, sachet, bag, or tube. Examples of water soluble packaging are available from Aquasol Corporation, located at 80 Thompson Street, North Tonawanda, N.Y. 14120.

Biocides control the growth of bacteria that produce enzymes exemplified by urease enzyme. Non-limiting examples of suitable biocides include: 3,5-Dimethyltetrahydro-1,3,5-thiadiazine-2-thione, Thiadiazine (Dazomet), 2,2-Dibromo-3-Nitrilopropionamide (DBNPA), 2-bromo-2-nitropropane-1,3-diol (Bronopol), 1,2-Thiazol-3-one (Isothiazolinone), tetrakis(hydroxymethyl)phosphonium sulfate (THPS), glutaraldehyde, formaldehyde, quaternary ammonium chlorides, chlorosulfamate and/or a blend of potassium monopersulfate, sodium chloride and sulfamic acid for the in-situ generation of chlorosulfamate.

The biocide is applied to achieve a concentration in the aqueous solution of the toilet holding from 20 ppm to 5000 ppm. The biocide can also be combined with the

Effervescing agents are often added to solid toilet treatments. Sodium bicarbonate or other carbon dioxide donors can be used in the presence of acid to release carbon dioxide when the composition is added to water. The effervescing disperses the ingredients so they do not clump and sink when the composition is introduced to the aqueous solution in the toilet holding tank.

Colorant is a dye used to color (typically a blue color) the solid composition, and/or may be used to impose a color to the aqueous solution in the toilet holding tank. One non-limiting example of a colorant that imposes color to aqueous solution in the toilet holding tank includes methylene blue. Titanium dioxide exemplifies a colorant suitable as a white colorant for a solid composition in the form of a tablet.

Fragrance is often added to provide a pleasant sent. The fragrance is often combined with amorphous silicon dioxide to absorb the oil based fragrance before adding to the solid toilet treatment. In liquid toilet treatments the fragrance can be added with or without surfactant or hydrotrope.

Binders are used to form tablets and slow the dissolution rate of the tablet when contacted by water and/or urine. Non-limiting examples of binders include polyethylene oxides (e.g. Polyox™ available through Dow Chemical) and/or Carbowax™ (PEG 8000 also sold by Dow Chemical).

Sequestrants and/or chelant can reduce or prevent scale buildup in urinals and piping. Sequestrant typically comprise phosphonic acid based compound. Non-limiting examples of sequestrants include: 1-hydroxyethylidene-1,1-diphosphonic acid, phosphonobutane carboxylic acid, aminotri(methylenephosphonic acid) and the like. Chelants typically comprise amino polycarboxylic acids. Non-limiting examples of chelants include: ethylene diamine tetraacetic acid, nitrilotriacetic acid and the like.

Dispersants can be used to disperse suspended solids and compromise deposits thereby making the deposit less adhesive to urinals and piping. The term polymer is used generically and includes polymers, copolymers and terpolymers. Non-limiting examples of suitable polymers are exemplified by the family of polymers sold under the trade name Acumer® by Dow Chemical.

The composition according to the first aspect of the invention substantially reduces or eliminates the release of offensive concentrations of ammonia even under conditions in which the urine and/or fecal contaminated water achieves a temperature of greater than or equal to 80° F., more preferably 90° F., and most preferably 100° F. for a period of at least 5 days, more preferably 6 days and most preferred 7 days.

In one embodiment, the invention comprises a method for treating portable toilets and urinals to reduce or eliminate ammonia odor, said method comprising the steps of: combining an aqueous solution comprising a source of hydroxyl amine and a source of organic acyl donor to produce a composition in the form of an aqueous solution; the normality based ratio of organic acyl donor to hydroxyl amine ranges from 1.5:1 to 0.5:1 respectively, more preferred 1.2:1 to 0.8:1 respectively, and most preferred 1:1 to 0.9:1 respectively, and introducing said composition into a toilet and/or urinal. The composition can be combined with other ingredients exemplified by the non-limiting examples comprising: biocide, colorant, fragrance, pH buffer, sequestrant, chelant, dispersant and surfactant before introducing into the toilet.

Dispensers can be used to release the composition in the form of an aqueous solution into the toilet holding tank or urinal. The dispenser typically comprises housing with an internal and replaceable cartridge and a drip tube to direct the said composition.

In another embodiment, the invention comprises a method for treating portable toilets and urinals to reduce or eliminate ammonia odor, said method comprising the steps of: combining an aqueous solution comprising a source of hydroxyl amine and a source of organic acyl donor to produce a composition in the form of an aqueous solution; the normality based ratio of organic acyl donor to hydroxyl amine ranges from 1.5:1 to 0.5:1 respectively, more preferred 1.2:1 to 0.8:1 respectively, and most preferred 1:1 to 0.9:1 respectively; drying the composition in the form of an aqueous solution to produce a solid composition; combining the solid composition with a pH buffer to produce a solid toilet treatment, and introducing said solid toilet treatment into a toilet and/or urinal. The solid toilet treatment can be combined with other ingredients exemplified by the non-limiting examples comprising: biocide, colorant, fragrance, sequestrant, chelant, dispersant and surfactant before introducing into the toilet.

In yet another embodiment, the invention comprises a method for treating portable toilets and urinals to reduce or eliminate ammonia odor, said method comprising the steps of: combining an aqueous solution comprising a source of hydroxyl amine and a source of organic acyl donor to produce a composition in the form of an aqueous solution; the normality based ratio of organic acyl donor to hydroxyl amine ranges from 1.5:1 to 0.5:1 respectively, more preferred 1.2:1 to 0.8:1 respectively, and most preferred 1:1 to 0.9:1 respectively; combining the composition in the form of an aqueous solution with amorphous silicon dioxide to effectively absorb the said aqueous solution to produce an absorbed composition; drying the absorbed composition to produce a solid composition; combining the solid composition, pH buffer, and a binder and producing a solid composition in the form of a tablet, and introducing said tablet into a urinal or portable toilet. The solid composition can be combined with other ingredients exemplified by the non-limiting examples comprising: biocide, colorant, fragrance, sequestrant, chelant and surfactant before producing a solid composition in the form of a tablet.

The tablet can be any geometric configuration or shape suitable for the release of the solid composition. The tablet may further comprise additives exemplified by biocide, fragrance, colorant, surfactant, sequestrant, chelant and dispersant. The tablet further comprises a binder that reduces the dissolution rate of the solid composition when the tablet is contacted by urine and/or water, and maintains the tablets structural integrity by allowing the tablet to shrink in size as it dissolves rather than fall apart or crumble. The said tablet can be applied to a urinal or water tank to slowly or periodically release the solid composition comprising at least the hydroxamic acid derivative and pH buffer over time.

In yet another embodiment, the invention comprises a method for treating portable toilets and urinals, said method comprising the steps of combining a source of hydroxyl amine, a source of water, and a source of organic acyl donor to produce a hydroxamic acid derivative having the general formula:

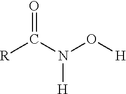

wherein R is selected from at least one: 1 to 6 carbon substituted or un-substituted alkyl group, 6 carbon substituted or un-substituted aromatic group, and 4 to 5 carbon substituted or un-substituted heterocyclic group; absorbing the hydroxamic acid derivative with amorphous silicon dioxide and drying to produce a solid composition; mixing the solid composition with a pH buffer and binder to produce a toilet treatment; forming the toilet treatment into a tablet, and applying the tablet to a toilet.

The tablet may further comprise additives exemplified by biocide, fragrance, colorant, surfactant, sequestrant, chelant and dispersant.

### DETAILED DESCRIPTION

As used herein, “aqueous solution in the toilet holding tank” is the liquid comprising water and/or urine in the holding tank that collects and holds the urine, fecal matter, and materials that are introduced through the toilet and/or urinal.

As used herein, “Hydroxamic acid derivatives” have the general formula:

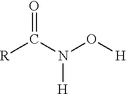

wherein R is selected from at least one: 1 to 6 carbon substituted or un-substituted alkyl group, 6 carbon substituted or un-substituted aromatic group, and 4 to 5 carbon substituted or un-substituted heterocyclic group.

Non-limiting examples of desirable hydroxamic acid derivatives include:

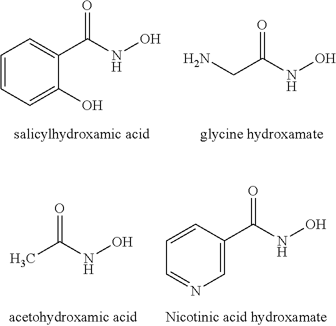

As used herein, “organic acyl donor” comprises carboxylic acids and derivatives of carboxylic acid. Examples of carboxylic acid derivatives include: acid chlorides, acid anhydrides, and esters. Non-limiting examples of organic acyl donors includes: acetyl chloride, acetic anhydride, acetic acid, ethyl acetate, salicylic acid, salicylic anhydride, benzoic acid, benzoic anhydride, benzoyl chloride, glycine, glycyl chloride, glycine anhydride, amino acids, nicotinic acid and the like.

As used herein, “source of hydroxyl amine” describes various forms of hydroxyl amine donor suitable for producing the hydroxamic acid derivatives exemplified by: hydroxyl amine free base, hydroxylamine hydrochloride, and hydroxylamine hydrosulfate.

As used herein, “water” describes an aqueous solution consisting of primarily water having the general formula H₂O. Water can include minerals, dissolved gases, solvents and other ingredients commonly used for the treatment of toilets exemplified by fragrance, colorant and the like.

As used herein, “source of water” describes the water used for preparing the composition resulting from combining a source of hydroxyl amine, a source of water and a source of organic acyl donor to produce the composition. The source of water may be water of good quality and clarity, and/or may be water provided by the source of hydroxyl amine. For example, hydroxyl amine free based comprises 50% water, and 50% sodium hydroxide comprises 50% water. If liquid versions of these additives are used, less or no additional water may be required. The source of water may be distilled water, demineralized water, or may be water comprising minerals and dissolved gases.

As used herein, “toilet treatment” can be in the form of a liquid, gel or solid. The toilet treatment comprises at least one hydroxamic acid derivative and pH buffer. The toilet treatment may further comprise fragrance, colorant, biocide, sequestrant, chelant, dispersant and surfactant. Solid toilet treatments may further comprise effervescing additives and/or binders.

As used herein, “tablet” can be any geometric configuration or shape suitable for the release of at least the hydroxamic acid derivative. The tablet may further comprise additives exemplified by pH buffer, biocide, fragrance, colorant, surfactant, as well as compounds that reduce the dissolution rate of the additives.

As used herein, “toilet” comprises at least the components or equipment involved with the collection and/or storage of urine and/or fecal matter. A toilet can be a portable toilet, a toilet in a mobile home or airplane, and a urinal.

As used herein, “treating portable toilets and urinals” describes the compositions and methods of the invention used to reduce or eliminate the undesirable odors emitted from a toilet holding tank and/or urinal. Portable toilets are commonly used for outdoor events and even in crowded stadiums to accommodate large numbers of people or to provide convenience in parks and other locations where people visit and gather. Typically these portable toilets are maintained on a weekly basis, wherein the holding tank is pumped out and replenished with water and a toilet treatment. A portable toilet may also include toilets used in mobile homes and airplanes. Undesirable odors include but may not be limited to ammonia, hydrogen sulfide and other reduced sulfur compounds.

Urease Inhibitor Test

To each of 8 flask, 200 ml of distilled water was combined with various concentrations of biocide, acetohydroxamic acid and pH buffer as illustrated in table 1. Sample 5 was used as a blank to represent biocide and pH buffer without the presence of urease inhibitor (acetohydroxamic acid). To each flask 9 ml of urine and 1.5 ml of liquefied fecal sample was added and mixed. The generation and release of ammonia was monitored by olfactory observation, and pH was monitored. On days 2, 3, and 4 an additional 9 ml of urine was added to the samples to represent daily loading. The flasks were heated to 80° F. for the entire period of the test.

| Sample # | 1 | 2 | 3 | 4 | 5 | 6 | 7 | 8 |
|---|---|---|---|---|---|---|---|---|
| Ingredient | | | | | | | | |
| BRONOPOL | .04 g | .04 g | .04 g | .02 g | .04 g | .04 g | .04g | .04g |
| Aceto- | .04 g | .04 g | .04 g | .04 g | | .08 g | .08g | .06g |
| hydroxamic | | | | | | | | |
| acid | | | | | | | | |
| Chloro- | | | .02 g | .04 g | | | | |
| sulfamate | | | | | | | | |
| Citric Acid | .10 g | .05 g | .10 g | .10 g | .10 g | .05g | .10g | .05g |
| | Day | Day | Day | Day | Day | Day | Day | Ammonia |
| Sample | 1 | 2 | 3 | 4 | 5 | 6 | 7 | Detection |
| | pH | pH | pH | pH | | | pH | |
| 1 | 1.60 | 2.30 | 3.40 | 5.40 | | | 6.49 | No |
| 2 | 2.00 | 3.00 | 4.70 | 6.55 | | | 7.22 | No |
| 3 | 1.63 | 2.25 | 3.50 | 5.28 | | | 5.94 | No |
| 4 | 1.55 | 2.16 | 3.65 | 6.75 | | | 7.90 | Yes-Strong |
| 5 | 1.62 | 2.30 | 3.50 | 6.10 | | | 7.27 | Yes-Strong |
| 6 | 1.97 | 2.98 | 4.70 | 6.81 | | | 7.57 | Yes-slight |
| 7 | 1.90 | 2.51 | 3.84 | 6.28 | | | 7.50 | Yes-Slight |
| 8 | 1.90 | 3.04 | 4.40 | 6.52 | | | 6.96 | No |

None of the samples were treated with fragrance or colorant to prevent any masking of ammonia and/or hydrogen sulfide odors. The results illustrate that there is a synergistic effect provided by combining biocide, pH buffer and hydroxamic acid derivative. Sample 5 with no acetohydroxamic acid had a strong ammonia odor that was detected by day 4. Sample 4 had lower bronopol concentration and a higher chlorosulfamate concentration. The higher concentration of chlorosulfamate may have oxidized some of the bronopol and/or acetohydroxamic acid, thereby compromising performance.

It is evident that combining hydroxamic acid derivative, biocide and pH buffer significantly improves the ammonia suppression of toilet treatment.

Producing Glycine hydroxamate (GH)

105 grams of hydroxyl amine hydrochloride was dissolved in 161.1 grams of distilled water by mixing in a 500 ml Erlenmeyer flask using a magnetic stirrer to form an aqueous solution. To the aqueous solution, 105.56 grams of glycine was incrementally added while mixing, allowing each increment to completely dissolve before additional glycine was added. After the final increment of glycine was added, the glycine hydroxamate (GH) solution was allowed to mix for approximately 60 minutes.

Producing GH Solids

The GH solution was added to 118 grams of HiSil 213 and mixed until the GH solution was absorbed by the amorphous silicon dioxide producing GH solids. The GH solids were placed in a ceramic dish and vacuum dried at approximately 60° C. and 20-25 inches Hg. The dried GH solids readily crumble into individual beads when mixed, and have an appearance ranging from beige to slight pink tint.

Tablets

All tablets were produced by combining the following ingredient:

10 grams of dried GH solids was treated and mixed with 2 grams of Mulberry fragrance oil,

12.5 grams of Polyox,

25 grams sulfamic acid, and

0.5 grams Solid Blue 38 obtained from Milliken.

The Polyox varied in molecular weight from 100,000 to 8,000,000. Each tablet is identified based on the polyox used.

All tablets were produced by adding the mixed ingredients listed, then decanting in to a 2″ diameter die. A Carver Press was used, applying a force of 20,000 lb_{f} to the die and sustaining the force until the applied force did not drift.

| | | Tablet Name | | | | |
|---|---|---|---|---|---|---|
| | | | | N-750/ | WSR | WSR |
| | Molecular | N-10 | N-80 | N-10 | 308 | 308/N-10 |
| Polyox | Wt | grams | grams | grams | grams | grams |
| N-10 | 100,000 | 12.5 | n/a | 3.5 | n/a | 9.0 |
| N-80 | 200,000 | n/a | 12.5 | n/a | n/a | n/a |
| N-750 | 300,000 | n/a | n/a | 9.0 | n/a | n/a |
| WSR 308 | 8,000,000 | n/a | n/a | n/a | 7.5 | 3.5 |

Dissolution Test

Each tablet was placed in a stainless steel sieve to support the tablet.

Water flow rate was set at 500 ml/min with a temperature range of 37° C. to 39° C.

N-10—Tablet was compromised in 20 minutes. The water stream eroded the tablet at the point of contact.

N-80—Eroded through in 40 minutes.

N-750/N-10—Provided excellent dissolution with relatively even dissolution across the tablet.

After two hours, approximately 70% of the tablet had dissolved.

WSR-308—Very even dissolution. However the tablet dissolved very slowly. After 3 hours, the tablet remained virtually intact with approximately 90% of the tablet remaining.

WSR-308/N-10—at the point of water contact, the tablet dissolved through while the remainder of the tablet remained virtually intact.

Testing Glycine Hydroxamate

To each of two flask, 200 ml of distilled water was combined with 0.04 grams of bronopol, 0.04 grams of either acetylhydroxamic acid or 0.04 grams of glycine hydroxamate, 0.10 grams of citric acid, 0.023 grams sodium bicarbonate, 0.027 grams of sulfamic acid, and 0.04 grams of mulberry fragrance. To each sample, 9 ml of urine and 1.5 grams of fecal matter was added and swirled. On days 2, 3 and 4, an additional 9 ml of urine was added. Temperature was held at approximately 80° F. After 7 days, there was no detectable odor of ammonia from either sample.

The test illustrate glycine hydroxamate performs comparable to acetylhydroxamic acid under test conditions.

I claim:

1. A method for treating toilets, said method comprising the steps of: combining a source of hydroxyl amine, a source of water and a source of organic acyl donor to produce a composition in the form of an aqueous solution; the normality based ratio of organic acyl donor to hydroxyl amine ranges from 1.5:1 to 0.5:1 respectively; combining said composition with a pH buffer to form a toilet treatment, and introducing said toilet treatment into a toilet.

2. The method in accordance with claim 1, further comprising the step of drying the composition to form a solid before combining with a pH buffer.

3. The method in accordance with claim 2, the method comprising combining the composition in the form of an aqueous solution with amorphous silicon dioxide before drying.

4. The method in accordance to claim 1, wherein the normality based ratio of organic acyl donor to hydroxyl amine ranges from 1.2:1 to 0.8:1.

5. The method in accordance to claim 1, wherein the normality based ratio of organic acyl donor to hydroxyl amine ranges from 1:1 to 0.9:1.

6. The method in accordance to claim 1, wherein the toilet treatment further comprising a biocide.

7. The method in accordance to claim 1, wherein the toilet treatment further comprising a fragrance.

8. The method in accordance to claim 1, wherein the toilet treatment further comprising a colorant.

9. The method in accordance to claim 1, wherein the toilet treatment further comprising a surfactant.

10. The method in accordance to claim 1, wherein the composition comprises hydroxamic acid derivative selected from at least one of acetohydroxamic acid, salicylhydroxamic acid, nicotinic acid hydroxamate and glycine hydroxamate.

11. A method for treating toilets, said method comprising the steps of: combining a source of hydroxyl amine, a source of water and a source of organic acyl donor to produce a hydroxamic acid derivative having the general formula: 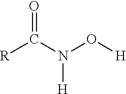
wherein R is selected from at least one: 1 to 6 carbon substituted or un-substituted alkyl group, 6 carbon substituted or un-substituted aromatic group, and 4 to 5 carbon substituted or un-substituted heterocyclic group; absorbing the hydroxamic acid derivative with amorphous silicon dioxide and drying to produce a solid composition; mixing the solid composition with a pH buffer and binder to produce a toilet treatment; forming the toilet treatment into a tablet, and applying the tablet to a toilet.

12. The method in accordance with claim 11, wherein the hydroxamic acid derivative comprises at least one of acetohydroxamic acid, salicylhydroxamic acid, nicotinic acid hydroxamate and glycine hydroxamate.

13. The method in accordance to claim 11, wherein the toilet treatment further comprising a biocide.

14. The method in accordance to claim 11, wherein the toilet treatment further comprising a fragrance.

15. The method in accordance to claim 11, wherein the toilet treatment further comprising a colorant.

16. The method in accordance to claim 11, wherein the toilet treatment further comprising a surfactant.

17. The method in accordance with claim 12, wherein the hydroxamic acid derivative comprises glycine hydroxamate.
